(19) United States
(12) Patent Application Publication
(10) Pub. No.: US 2012/0271357 A1
(43) Pub. Date: Oct. 25, 2012

(54) TOOLS AND METHODS FOR THE PREPARATION OF THE FACET JOINT

(75) Inventors: Amy L. Arthur, Mountain View, CA (US); Mojan Goshayeshgar, Atherton, CA (US); Mehboob Kadri, Union City, CA (US); Craig E. Lauchner, Mountain View, CA (US); Newton H. Metcalf, JR., Memphis, TN (US); Neil S. Sasaki, Sunnyvale, CA (US)

(73) Assignee: Warsaw Orthopedic, Inc., Warsaw, IN (US)

(21) Appl. No.: 13/091,265

(22) Filed: Apr. 21, 2011

(51) Int. Cl. A61B 17/88

(52) U.S. Cl. 606/279

(57) ABSTRACT

Methods of fusing a facet joint that include inserting a cannula with a first end of the cannula at the facet joint and a second end positioned away from the facet joint. One or more tools may be inserted through the cannula to contact against and treat one or both vertebral members to enlarge the facet joint. An osteogenic material is then inserted into the enlarged facet joint to facilitate fusion of the first and second vertebral members.

### BACKGROUND

Each vertebral member includes an anterior arch and a posterior arch. The posterior arch includes two pedicles and two laminae that join together to form the spinous process. Two transverse processes are laterally positioned at the transitions from the pedicles to the laminae. Both the spinous process and transverse processes provide for attachment of fibrous tissue, including muscle. Two inferior articular processes extend downward from the junction of the laminae and the transverse process. Further, two superior articular processes extend upward from the junction. The articular processes of adjacent vertebrae form the facet joints with the inferior articular process of one vertebral member engaging with the superior articular process of the vertebral member below.

The facet joints permit motion between individual vertebral members. The facet joints are gliding joints because the articular surfaces glide over each other. Fusing of the facet joint may be appropriate in various situations. Facet fusion is increasingly used as a treatment for facet-mediated pain, as well as to supplement posterior fixation. Facet fusion often involves destruction of the facet by decorticating the opposing articulating surfaces and packing osteogenic material into the space between the articular processes. The preparation of the facet joint may be performed percutaneously which offers various benefits over an open approach.

Tools are needed to provide access to and treat the facet joints of the vertebral members. Because the facet joints are generally small as compared to some other features of the vertebral members (e.g., the intervertebral space formed between adjacent vertebral members), new tools are required that fit down a relatively small cannula and into the facet joint. Tools used for treating other areas of the spinal column may not be applicable with methods for treating the facet joint.

### SUMMARY

The present application is directed to methods and devices for fusing a facet joint. Various tools are disclosed that are inserted into the facet joint and treat one or both of the vertebral members. A cannula may be used to provide access for the tool into the facet joint. Once the facet joint has been treated, an osteogenic material may be inserted into the enlarged facet joint to facilitate fusion of the first and second vertebral members.

One aspect is directed to a method of fusing a facet joint that includes percutaneously inserting a cannula into a patient with a first end of the cannula at the facet joint and a second end of the cannula positioned outward from the patient. The method includes inserting a first tool through the cannula and moving a treating section of the tool outward beyond the first end of the cannula and moving the treating section of the first tool and contacting against at least one of the vertebral members and enlarging the facet joint. The first tool is removed through the second end of the cannula and a second tool is inserted into the second end of the cannula. A treated section of the second tool is moved outward beyond the first end of the cannula. The method includes moving the treating section of the second tool and enlarging the facet joint and removing the second tool through the second end of the cannula. The method further includes inserting an osteogenic material into the enlarged facet joint.

Another aspect is directed to a method of fusing a facet joint formed by first and second vertebral members that includes inserting a cannula into a patient with a first end of the cannula that includes a cutting edge at the facet joint and a second end of the cannula spaced away from the facet joint. The method includes moving the cannula and contacting the cutting edge against the first and second vertebral members and removing first portions of the first and second vertebral members with the cutting edge. The method includes inserting a tool through the cannula and moving a treating section of the tool outward beyond the first end of the cannula and moving the treating section of the tool against the first and second vertebral members and removing second sections of the first and second vertebral members. The method includes removing the tool through the second end of the cannula, and inserting an osteogenic material into the facet joint and against the first and second vertebral members.

Another aspect is directed to a method of fusing a facet joint formed by first and second vertebral members including percutaneously inserting a cannula into a patient with a first end of the cannula at the facet joint and a second end of the cannula positioned outward from the patient. The method includes inserting a tool into the second end of the cannula with a treating section of the tool in a retracted orientation and moving the treating section along the cannula and away from the second end. The method includes deploying the tool and moving the treating section of the tool to a second extended orientation, and contacting the treating section of the tool in the extended orientation against at least one of the first and second vertebral members and removing sections of at least one of the first and second vertebral members. The method further includes returning the tool to the retracted orientation, and removing the tool through the second end of the cannula while the tool is in the retracted orientation.

The various aspects of the various embodiments may be used alone or in any combination, as is desired.

### BRIEF DESCRIPTION OF THE DRAWINGS

FIG. 1 is a side view of first and second vertebral members.

FIG. 2 is a schematic sectional view taken along line II-II illustrated in FIG. 1.

FIG. 3A is a perspective view of a cannula.

FIG. 3B is a perspective view of a tool.

FIG. 3C is a schematic view of a handle.

FIG. 4 is perspective view of a reamer tool.

FIG. 5 is a side view of a burr tool.

FIG. 6 is a side view of a spade tip tool.

FIG. 7 is a side view of a countersunk tip tool.

FIG. 8 is a perspective view of a trephine tip tool.

FIG. 9 is a partial perspective view of a first end of a trephine tip tool.

FIG. 10 is an end view of a trephine tip tool.

FIG. 11 is a side view of an auger tool.

FIG. 12 is a perspective view of a broach tool.

FIG. 13A is a front view of a rasp tool.

FIG. 13B is a side view of the rasp tool of FIG. 13A.

FIG. 14 is a partial perspective view of a first end of a chisel tip tool.

FIG. 15 is a perspective view of a curette tool.

FIG. 16 is a partial perspective view of a head at the first end of the curette tool of FIG. 15.

FIG. 17 is a side view of a saw tool.

FIG. 18 is a perspective view of a brush tool.

FIG. 19 is a perspective view of an abrasive surface tool.

FIG. 19A is a perspective view of a cannula.

FIG. 20 is a side schematic view of a deployable tool.

FIG. 21A is an end view of the deployable tool of FIG. 20 in a first orientation.

FIG. 21B is an end view of the deployable tool of FIG. 20 in a second orientation.

FIG. 22 is a side view of a portion of a deployable tool.

FIG. 23 is a sectional schematic view of a portion of a deployable tool.

FIG. 24 is a sectional schematic view of a portion of a deployable tool.

FIG. 25A is a perspective view of a deployable tool in a first orientation with a portion of the housing cut away.

FIG. 25BA is a perspective view of a deployable tool in a second orientation with a portion of the housing cut away.

FIG. 26A is a perspective view of a deployable tool in a first orientation with a portion of the housing cut away.

FIG. 26B is a perspective view of a deployable tool in a second orientation with a portion of the housing cut away.

FIG. 27 is a sectional schematic view of a deployable tool.

FIG. 28 is a sectional schematic view of a deployable tool.

FIG. 29 is a sectional schematic view of a deployable tool.

FIG. 30 is a perspective view of a deployable tool.

FIG. 31 is a partial perspective view of a cannula.

FIG. 32 is a partial perspective view of a cannula.

FIG. 33 is a side view of a cage.

FIG. 34 is a side schematic view of a mesh casing.

### DETAILED DESCRIPTION

The present application is directed to various tools and methods for accessing the facet joint, treating the surfaces of the facet joint formed by the articular processes, and insertion and placement of osteogenic material into the treated facet joint.

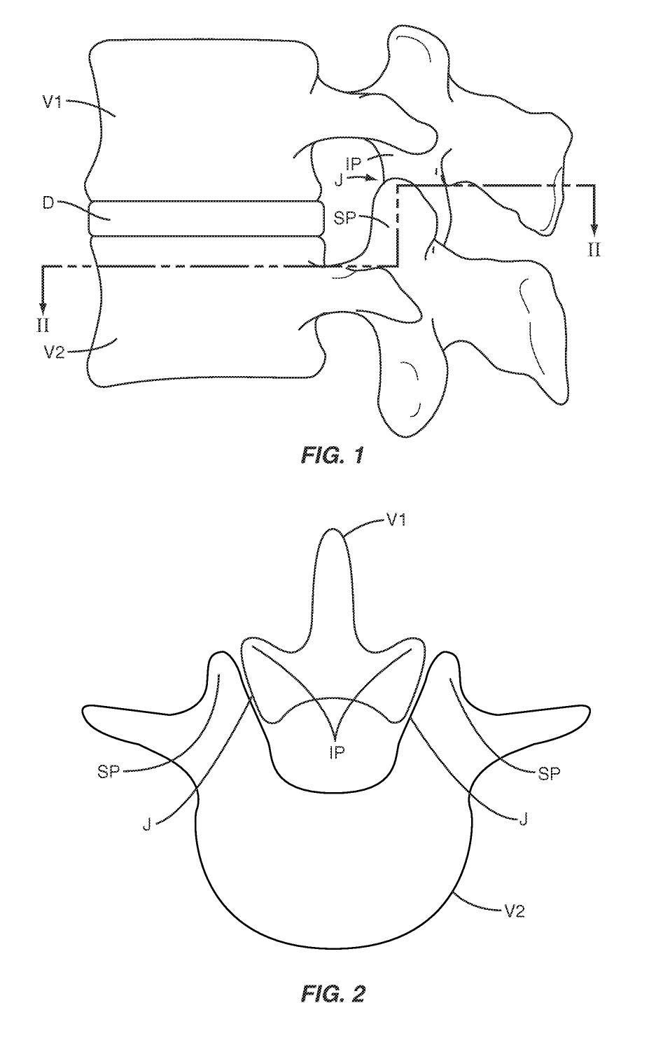

FIG. 1 illustrates a lateral view of two vertebral members V1, V2 and an intervertebral disc D disposed therebetween. The section view shown in FIG. 2 is depicted according to the section line labeled II-II in FIG. 1. FIG. 2 illustrates the facet joints J formed between the inferior articular process IP of the superior vertebral member V1 and the superior articular process SP of the inferior vertebral member V2.

The facet joints J may be accessed using a cannula 10. In one embodiment, the cannula 10 provides for percutaneous access to the facet joint J without the need for an open procedure. The approach angle into the facet joint J may vary depending upon the specific application. In one embodiment, the approach is to enter at the mid-point of the facet joint J (mid-plane superior to inferior and mid-plane medial to lateral). Other approaches may include but are not limited to at the superior aspect of the joint and mid-plane medial to lateral, at the inferior aspect of the joint and mid-plane medial to lateral, and across the joint.

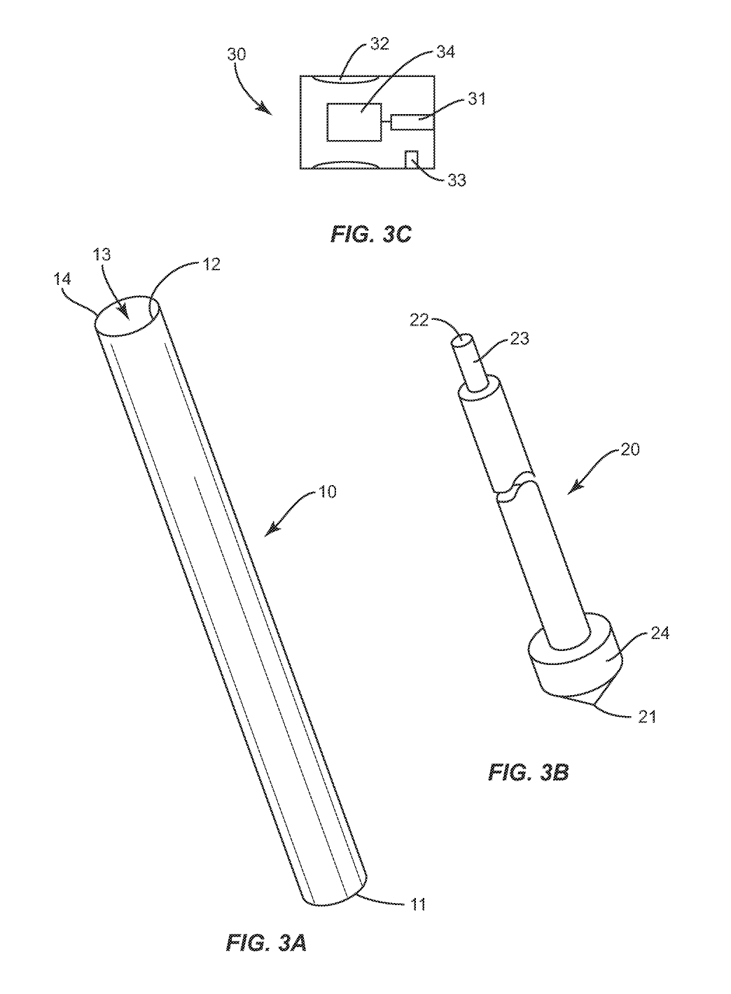

FIGS. 3A, 3B, and 3C illustrate the basic components of the devices used for accessing and treating the facet joint J. These include a cannula 10, tool or stylet 20, and a handle 30.

The cannula 10 includes a first end 11 that is positioned at the facet joint J, and an opposing second end 12. The cannula 10 includes a length measured between the ends 11, 12 that positions the second end 12 on the exterior of the patient when the first end 11 is at the facet joint J. The cannula 10 includes a hollow interior 13 for insertion of tools 20 for treating the facet joint J. The cross-sectional size and/or shape of the cannula 10 may vary with examples including but not limited to circular as illustrated in FIG. 3A, rectangular, and oblong. The cross-sectional shape and size may be consistent or may vary along the length. In one embodiment, the cross-sectional size gradually tapers from an enlarged size at the second end 12 to a reduced size at the first end 11. The cannula 10 may have various sizes and lengths. Embodiments include the cannula 10 having a first end 11 with outer diameters between about 3 mm and 10 mm. The diameter may be the same along the length, or may vary with the second end 12 having a different diameter. The length of the cannula may vary, with embodiments including a length of between 5 cm and 20 cm.

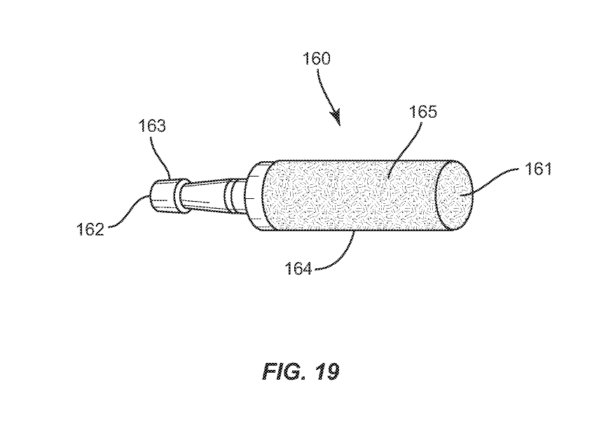

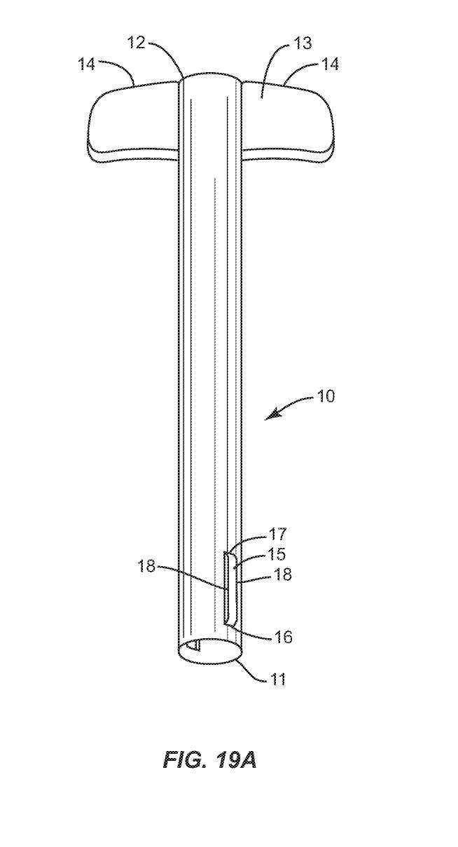

The cannula 10 may also include one or more striking surfaces 14 at the second end 12. The striking surfaces 14 may be used with an impact tool (e.g., hammer) to apply a force to move the cannula 10 into the patient. Embodiments include the striking surfaces 14 being formed by the second end 12, and by a handle 13 (see FIG. 19A). The handle 30 may also form another striking surface.

The tool 20 is sized to fit into the cannula 10 and is configured for treating the facet joint J. The tool 20 generally includes an elongated shape with opposing ends 21, 22. A mount 23 is positioned in proximity to the second end 22 to attach to the handle 30. A treating portion 24 is positioned at or in proximity to the first end 21 and is configured to contact against and remove portions of one or both of the inferior articular process IP of the superior vertebral member V1 and the superior articular process SP of the inferior vertebral member V2. The tool 20 may be configured for one or both of linear and rotary motion for treating the vertebral members V1, V2. For rotary motion tools, the tools may be configured for rotation in a first direction (e.g., clockwise), a second direction (e.g., counter-clockwise), or both directions.

The length of the tool 20 is generally longer than the cannula 10 to position the treating portion 24 at or outward from the first end 11 while the mount 22 is at or outward from the second end 22. Alternatively, the length of the tool 20 may be shorter than the cannula 10. In some embodiments, the cannula 10 includes one or more windows 15 through which the treating portion 23 accesses the facet joint J.

The handle 30 attaches to the tool 20. A receptacle 31 is sized to receive the mount 22 for attachment with the tool 20. The handle 30 may be permanently attached to the mount 22, or may be removed for detachment to other tools 20. One or more grips 32 may be placed on the exterior of the handle 30 for grasping by the surgical personnel. The grips 32 may include indented sections, knurled surfaces, and various other features that facilitate contact. An end of the handle opposite from the receptacle 31 may form a striking surface for receiving impact from a hammer.

The handle 30 may also provide a means for applying a force to the tool 20. The handle 30 may allow for gripping by the surgical personnel who manually applies the necessary power to operate the tool 20. FIG. 3C illustrates a handle 30 that includes a drive system 34 and trigger 33. The drive system 34 provides linear or rotary motion to the tool 20. The drive system 34 may provide power in a single direction (e.g., clockwise rotary motion), or in multiple directions. The drive system 34 may also operate at varying speeds depending upon the application. The trigger 33 provides for the medical personnel to operate the tool 20 at the various settings. The drive system 34 may be powered by various sources, including one or more batteries and any standard electrical source, such as 110 volt, 60 cycle power sources, with or without a transformer to reduce the voltage as necessary.

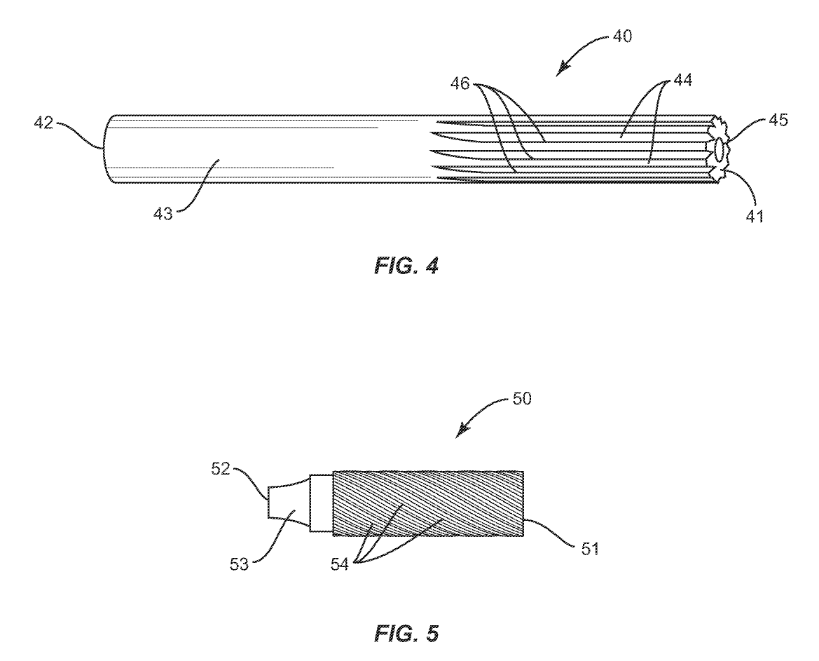

Various tools 20 may be configured to treat the vertebral members V1, V2 using rotary motion. One rotary tool 20 includes a reamer 40 as illustrated in FIG. 4. The reamer 40 generally includes an elongated shape with a first end 41 and opposing second end 42. A mount 43 is configured to attach with the handle 30. A number of flutes 44 are positioned at the first end 41 and are separated by intermediate troughs. Cutting edges 46 are positioned along one or more of the flutes 44, and may be undercut. FIG. 4 includes a set of straight flutes 44 that are each aligned parallel with a longitudinal axis of the reamer 40. Other embodiments may include helical flutes 44 arranged in either clockwise or counter-clockwise orientations.

The first end 41 may be flat and aligned at various angled orientations relative to the longitudinal axis. The first end 41 may also include a tapered shape. An opening 45 may extend through the length of the reamer 40 and is configured for receiving a guide wire.

Another rotary tool includes a burr 50 as illustrated in FIG. 5. The burr 50 includes a first end 51, second end 52, and a mount 53. A number of helical cutting edges 54 extend inward from the first end 51. FIG. 5 includes each of the edges 54 helically wound in a common direction. Additional cutting edges 54 may extend inward from the first end 51 in an opposing helical direction. The first end 51 is illustrated in FIG. 5 as being substantially flat, although other shapes may also be possible. The cross-sectional shape of the treating section that includes the cutting edges 54 may be circular (as illustrated in FIG. 5), semi-spherical, or conical with a reduced width at the first end 51 that tapers outward towards the second end 52. FIG. 5 includes the cutting edges 54 being helical, although other embodiments may include straight cutting edges 54.

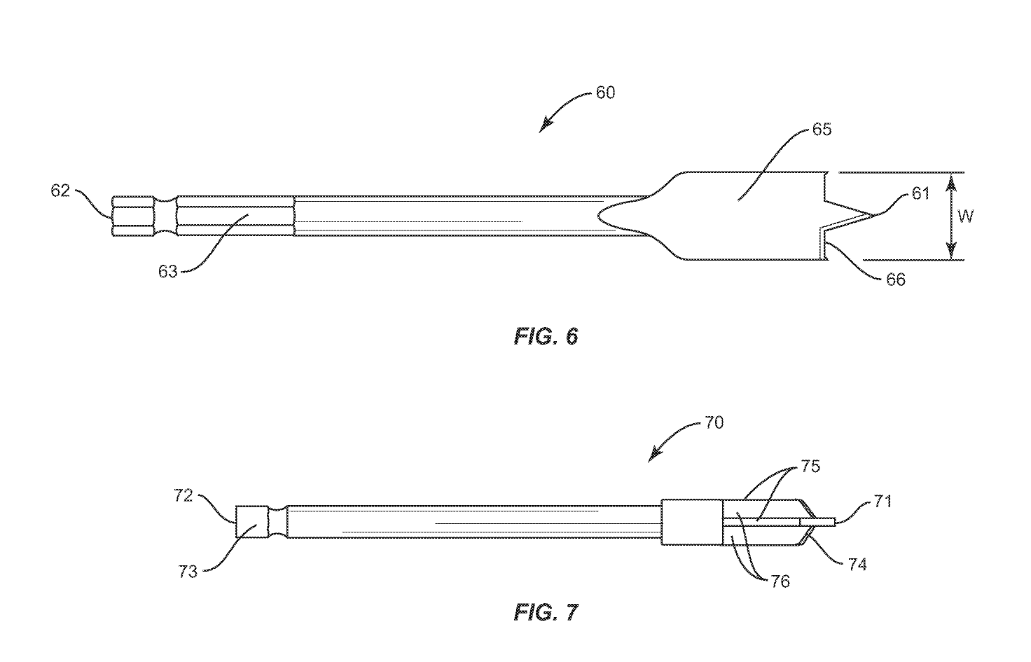

A spade tip tool 60 illustrated in FIG. 6 is another tool configured for rotary motion. The spade tip 60 includes an enlarged flat section 65 at the first end 61 that includes a substantially rectangular portion and a tapered tip that terminates at the first end 61. Leading edges of the rectangular section include cutting edges 66 that may also extend along the tapered tip. The flat section 65 transitions into a circular shaft that includes a mount 63 at a second end 62.

FIG. 7 includes a countersunk tip tool 70 that includes a series of extensions 75 that extend longitudinally at the first end 71 and are separated by troughs 76. The extensions 75 include tapered ends that terminate at a tip at the first end 71. The tool 70 includes an opposing second end 72 and a mount 73 for attachment to the handle 70.

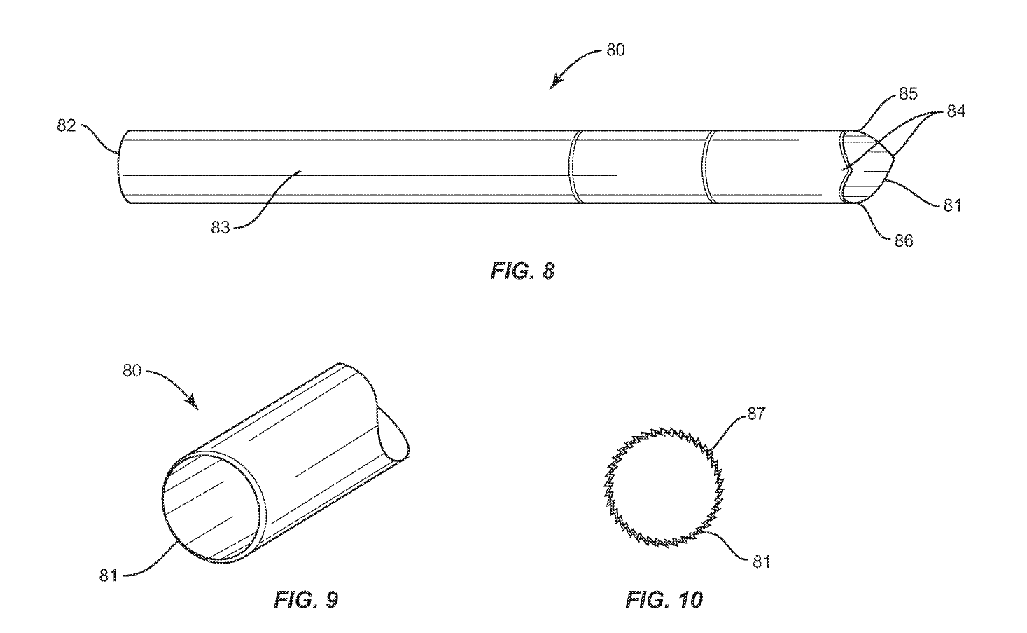

FIG. 8 illustrates a trephine tip tool 80 with a first end 81 forming a cylindrical cutting edge that faces longitudinally outward. The embodiment of FIG. 8 includes the cutting edge having a pair of tips 84 positioned on opposing sides of a hollow interior space. Opposing blade sections 85, 86 angle axially backward away from the tips 84. FIG. 9 illustrates an embodiment with a cylindrical cutting edge that lies within a plane. The cutting edge may be angled with respect to the longitudinal axis of the tool 80. In one specific embodiment, the cutting edge is substantially perpendicular to the longitudinal axis. The tool 80 further includes a shaft with a mount 83 and terminates at a second end 82.

The various cutting edges may include a tapered blade that faces axially outward to engage with the vertebral members V1, V2. The cutting edges may also include teeth 85 that extend around a limited portion or entirety of the exposed first end 81. FIG. 10 illustrates on embodiment of the teeth 87 positioned around the entirety of the first end 81.

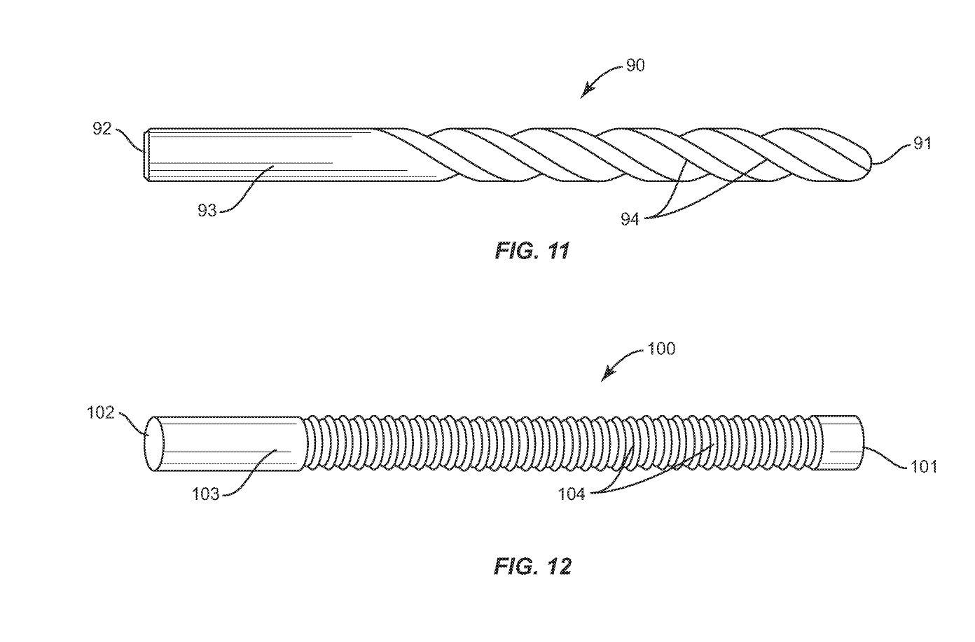

Another embodiment includes an auger 90 as illustrated in FIG. 11. The auger 90 includes an elongated shape with opposing first and second ends 91, 92 and a mount 93. Helical threads 94 wrap around the body of the auger 90 and extend from the first end 91 towards the second end 92. The threads 94 may include a variety of sizes and be arranged at various pitches. FIG. 11 includes a single thread wrapping around the tool 90, although the tool 90 may also include multiple threads. The first end 91 may include a tapered tip.

Additional tools 20 may be sized to fit through the cannula 10 and operate with linear motion to treat the vertebral members V1, V2. A broach 100 as illustrated in FIG. 12 is one type of linear motion tool. The broach 100 includes an elongated shape with opposing ends 101, 102 and a mount 103. A series of circumferential teeth 104 extend around the broach 100. Each of the teeth 104 include a leading face that faces towards the first end and includes a cutting edge. A gullet is positioned between each row of teeth 104 to capture the material removed from the vertebral members V1, V2. The teeth 104 may further include a cutting edge on the trailing face that faces towards the second end 102. Each of the rows of teeth 104 along the length may include substantially the same radial height, or may include tapering heights with the teeth 104 closest to the first end 101 having a different radial height than those closest to the second end 102.

Although FIG. 12 includes the teeth 104 being spaced away a distance from the first end 101, other embodiments position the teeth at the first end 101. Teeth 104 may also be placed at other axial locations along the broach 100. FIG. 12 also includes the teeth 104 extending completely around the circumference of the tool 100. Teeth 104 may extend around less than the entire circumference. In one embodiment, teeth 104 are positioned on limited sections of the broach 100. In one specific embodiment, a first section of teeth 104 extend on a first side of the tool 100, and a second section of teeth 104 extend on an opposing second side of the tool 100 with two intermediate sections between the teeth 104 being devoid of teeth.

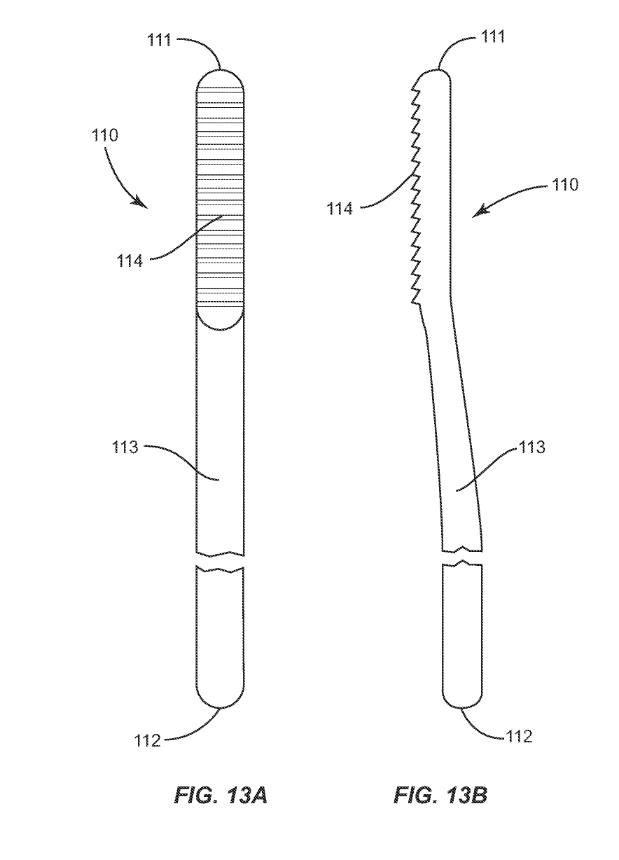

FIGS. 13A and 13B illustrate a rasp tool 110 for use with linear motion to treat the vertebral members V1, V2. The tool 110 includes an elongated shape with a series of teeth 115 positioned on a face 114 at the first end 111. Each of the teeth 115 includes one or more cutting edges. In one embodiment, each of the teeth 115 are individual and spaced apart from each other. The teeth 115 may also be aligned in rows as illustrated in FIGS. 13A and 13B. Each row includes one or more cutting edges with intermediate indetents positioned between adjacent rows. The rows are transverse to the longitudinal axis of the tool 110 as illustrated. In one specific embodiment, the rows are perpendicular to the longitudinal axis of the tool 110. The teeth 115 may also include tapered spikes that extend outward from the face 114 and terminate at a sharpened point. These teeth 114 may be positioned at various patterns across the face 114.

The face 114 is formed at the first end 111 and extends between the lateral sides of the tool 110. The face 114 may be substantially flat, or may be rounded. The tool 110 further includes an elongated body with an opposing second end 112 and a mount 113.

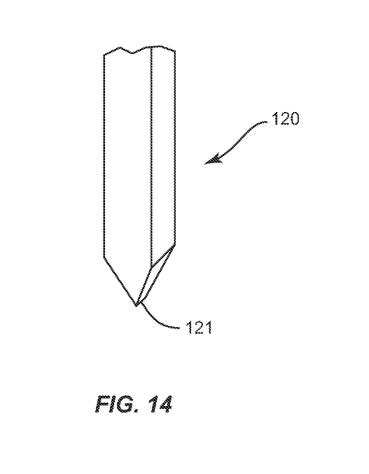

Another linear motion tool includes a chisel tip 120 as illustrated in FIG. 14. The chisel tip tool 120 includes a tapered tip 121 with a leading cutting edge that contacts against the vertebral members V1, V2.

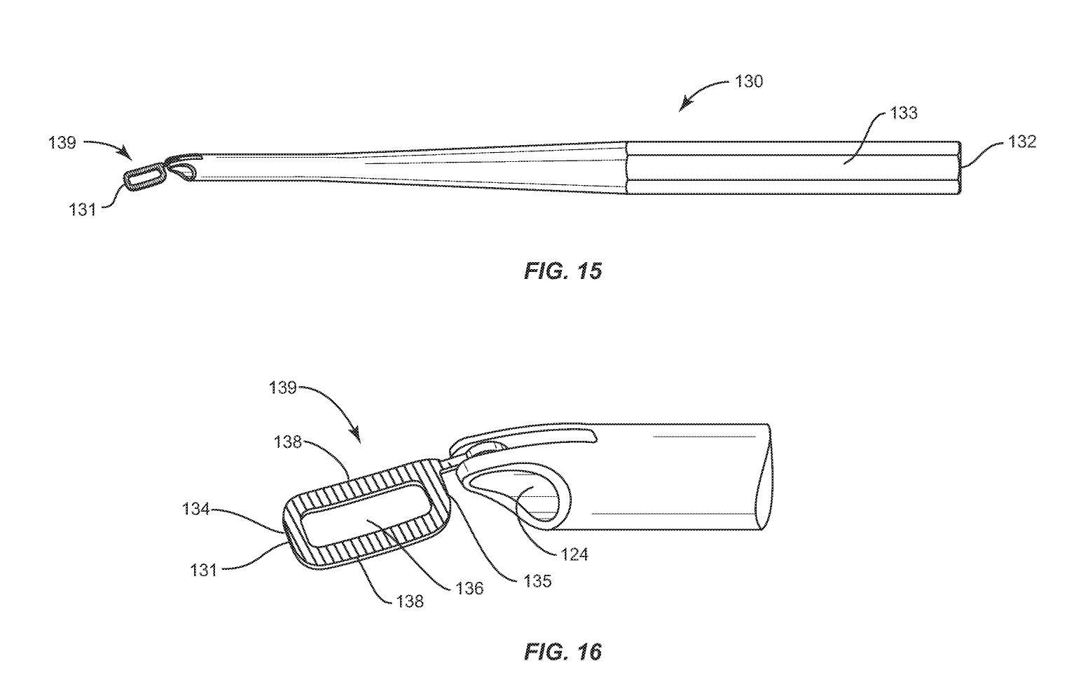

A curette 130 as illustrated in FIGS. 15 and 16 is another linear motion tool. As illustrated in FIG. 15, the curette 130 includes an elongated shape with first and second ends 131, 132 and a mount 133. A head 139 is positioned at the first end 131 for contacting against the vertebral members V1, V2.

As illustrated in FIG. 16, the head 139 includes a nose 134, a tail 135, and lateral sections 138 that form an opening 136. The opening 136 may extend completely through the head 139, or may include a bottom. One or more cutting edges are positioned along the head at one or more of the nose 134, tail 135, and lateral sections 138. The cutting edges are exposed to treat the vertebral member. Cutting edges may be positioned along a single side of the head 139 for treating a single vertebral member V1, V2, or may also be positioned along the opposing side of the head 139 for treatment of two vertebral members.

The head 139 may have various shapes, including but not limited to rectangular as illustrated in FIGS. 15 and 16, round, and oblong. FIGS. 15 and 16 include the head completely enclosing the opening 139. Other embodiments may include one or more inlets through the head 139 and into the opening 136 (i.e., the opening 136 is not fully enclosed within the head 139).

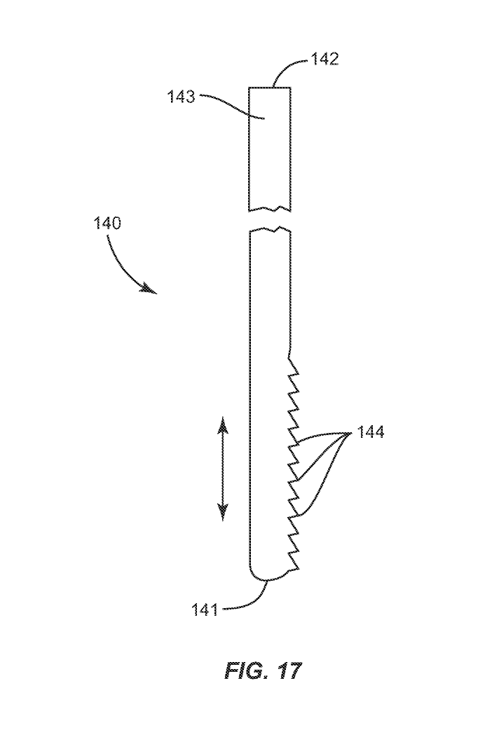

Another linear cutting tool is a saw blade 140 as illustrated in FIG. 17. The saw blade 140 includes opposing first and second ends 141, 142 and a mount 143. Teeth 144 are positioned between the ends 141, 142. The teeth 144 may be positioned at the first end as illustrated in FIG. 15, or may be spaced away from the first end 141. The teeth 144 may be configured for treating the vertebral members V1, V2 during linear motion in a first direction, a second direction, or both.

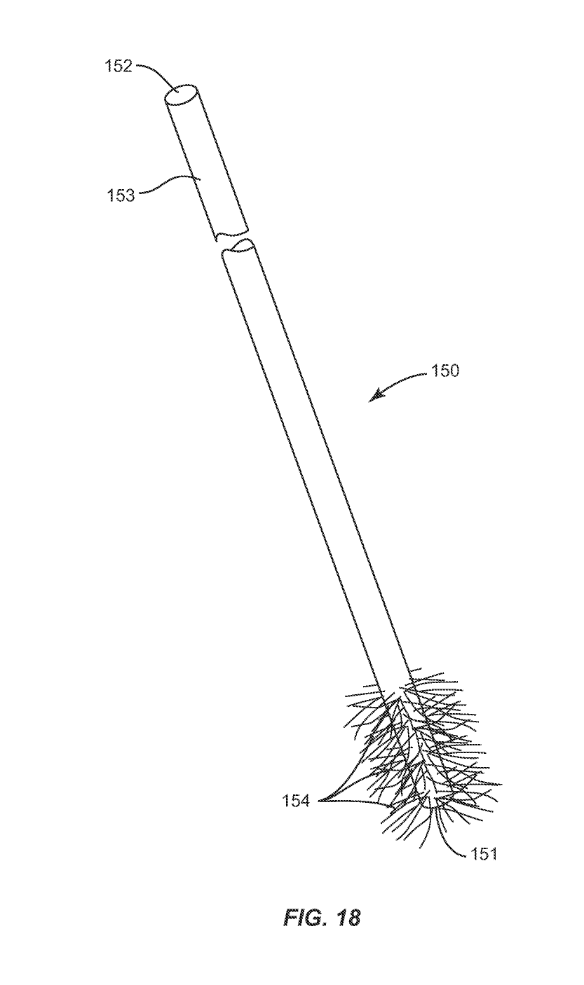

A brush 150 as illustrated in FIG. 18 includes an elongated shaft with opposing ends 151, 152, and a mount 153. Bristles 154 are attached to and extend outward from the shaft at the first end 151. The bristles 154 are constructed of a rigid material, including but not limited to titanium, stainless steel, and cobalt chrome. The various bristles 154 may be constructed of the same or different materials.

The bristles 154 terminate at an exposed end that contact against the vertebral members V1, V2. The bristles 154 may remain substantially rigid without deformation during contact with the vertebral members V1, V2. Alternatively, the bristles 154 may flex during contact. The amount of flexing may be the same in both directions of linear motion. Alternatively, the bristles 154 may be constructed to flex a greater amount during movement in one direction than in the second direction. In one embodiment, the bristles 154 include a curved or bent shape that provides for the different amounts of flexing depending upon the direction of linear motion. In one embodiment, the width of the brush 150 is greater than the inner diameter of the cannula 10. This causes the bristles 154 to flex as the tool 150 moves through the cannula 10.

FIG. 19 illustrates an abrasive surface tool 160 having a working section 164 with an abrasive exterior surface. The working section 164 is sized to contact against and treat the vertebral members V1, V2. The size and shape of the working section 164 may vary. One example is shown in FIG. 19 having a cylindrical shape that forms the first end 161 of the tool 160. The working section 164 is attached to a shaft that includes the mount 163 and second end 162.

The working section 164 may include an interior body with an abrasive coating 165. The coating 165 can be applied using known metallic coating processes including Ion Bean Assisted Deposition (IBAD) process or a plasma coating type process. Other known coating processes and techniques may be used depending on the interior body and the specific metal coating used including, among others, cathodic arc deposition, sputter deposition, ion beam induced deposition, atmospheric plasma spray, arc spray, cold spray, plasma spray process, high velocity oxy-fuel (HVOF), vacuum plasma spraying (VPS), ion beam sputtering and pulsed laser deposition. The coating 165 may be comprised of a biocompatible metal such as titanium (Ti), Gold (Au), Stainless Steel, Cobalt Chrome, Tantalum, Platinum, Tungsten, Silver, Palladium, as well as any mixture, composite, combination an/or alloy of the aforementioned biocompatible metallic materials. In one specific embodiment, the abrasive coating 165 is a titanium sputter coating.

Some of the tools described above as being either for linear or rotary motion may also have application for both types of motion. For example, the trephine tip tool 80 described above for rotary motion may also be used with linear motion tool. Likewise, some of the linear motion tools 20 may be used with rotary motion. The brush tool 150 illustrated in FIG. 18 may be used with rotary motion to treat the vertebral members V1, V2.

Many of the features described above for the different tools 20 may also be applied to the cannula 10. The cannulas 10 may also be configured to treat the vertebral members V1, V2 with linear or rotary motion. These cannulas 10 may be used by themselves (i.e., without a tool 20), or may be used in combination with a tool 20. In these embodiments, the first end 11 of the cannula 10 includes structure to treat the vertebral members V1, V2. Alternatively or in addition, the cannula 10 may include features away from the first end 10 for treating the vertebral members V1, V2.

The cannula 10 may be configured for treating the vertebral members V1, V2 with rotary motion. The exterior of the cannula 10 may include cutting edges and flutes 44 as illustrated in the reamer 40 of FIG. 4, cutting edges 54 extending in a helical pattern as illustrated with the burr of FIG. 5, and helical threads 94 as illustrated in the auger 90 of FIG. 11. Another example includes a cannula 10 with a trephine tip as illustrated in the embodiments of FIGS. 8, 9, and 10. The cannula 10 includes a hollow interior with the first end 12 having one or more cutting edge for treating one or both vertebral members V1, V2. The first end 12 may also include teeth 85 that can further treat the vertebral members.

The cannula 10 may also include features for use with linear motion to treat the vertebral members V1, V2. These features may include teeth 104 as illustrated in the broach 100 of FIG. 12, or teeth 115 of the rasp 110 in FIGS. 13A, 13B. The cannula 10 may also include bristles 154 that extend outward as illustrated in FIG. 18.

The cannula 10 may also include independent features that are applicable for use with linear and/or rotary motion. The cannula 10 may include one or more windows 15 as illustrated in FIG. 19A. The windows 15 include a leading section 16, trailing section 17, and lateral sections 18. Cutting edges 19 extend along one or more of the sections 16, 17, 18 and contact against and treat the vertebral members V1, V2 during movement of the cannula 10 in the facet joint J. The cutting edges 19 may be positioned to engage with the vertebral members V1, V2 during linear and/or rotary motion of the cannula 10.

One or more of the sections 16, 17, 18 may extend radially outward from a longitudinal axis of the cannula 10 a further distance than the remainder of the window 15. This positioning further exposes the cutting edges 19 to engage with the vertebral members V1, V2. In one embodiment for use with linear motion, the trailing section 17 radially extends outward a greater distance from the axis. In another embodiment that utilizes rotary motion, one of the lateral sections 17 extends radially outward.

A handle 13 may be positioned on the cannula at the second end 12. The handle 13 may include a gripping surface to facilitate placement into the patient and to apply a force for rotary and/or linear motion during treatment of the vertebral members V1, V2.

Because of the relative small working space available for the processing of the facet joints J, tools 20 may be configured to be deployed between a first reduced-sized orientation and a second enlarged-sized orientation. These tools 20 are positioned in the first orientation during insertion into the cannula 10. Once positioned, the tools 20 are configured to deploy to the enlarged second orientation for treating the vertebral members V1, V2. Once completed, the tools 20 can be returned to the first position for removal through the cannula 10.

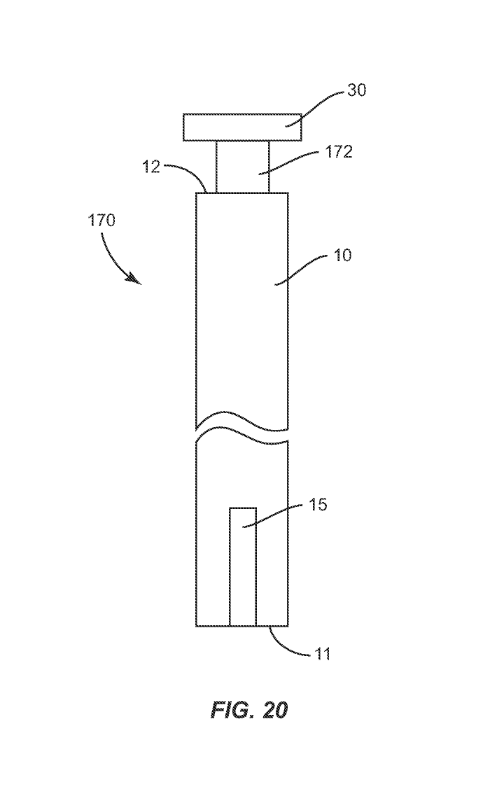

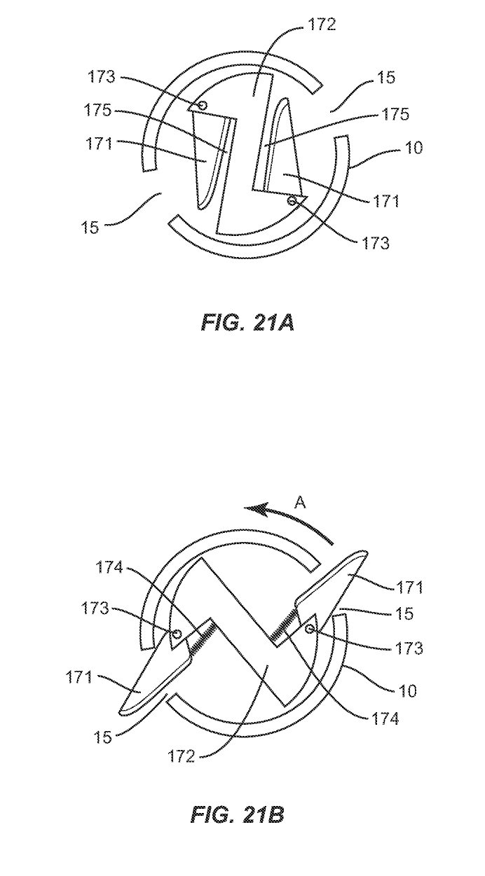

FIGS. 20, 21A, and 21B include a device with a deployable tool 170 that includes a shaft 172 that extends through the cannula 10. One or more teeth 171 are connected by hinges 173 to the shaft 172. Biasing members 174 are positioned between the shaft 172 and each of the teeth 171 to bias the teeth 171 radially outward away from the shaft 172. The cannula 10 includes a pair of windows 15. The windows 15 may be positioned at the first end of the shaft 11, or may be spaced away from the first end 11.

As illustrated in FIG. 21A, the shaft 172 includes pockets 175 that each receives one of the teeth 171. The teeth 171 and shaft 172 overlap forming a substantially circular cross-sectional shape. The teeth 171 are held in the retracted first position as illustrated in FIG. 21A due to contact with the inner surface of the cannula 10.

Rotation of the shaft 172 in the direction of arrow A causes deployment of the teeth 171 through the windows 15. During rotation, the tips of the teeth 171 align with the window 15. The force of the biasing members 174 in a radial outward direction causes the teeth 171 to pivot about the hinges 173 and extend outward through the windows 15 to a deployed orientation as illustrated in FIG. 21B. The deployed orientation positions the sharpened tips of the teeth 171 radially outward beyond the cannula 10. The shaft 172 and the cannula 10 are rotated together to treat the vertebral members V1, V2. Once complete, the shaft 172 is rotated in a direction opposite to that of arrow A. This causes the teeth 171 to contact against the edges of the windows 15 and pull radially inward into the interior of the cannula 10.

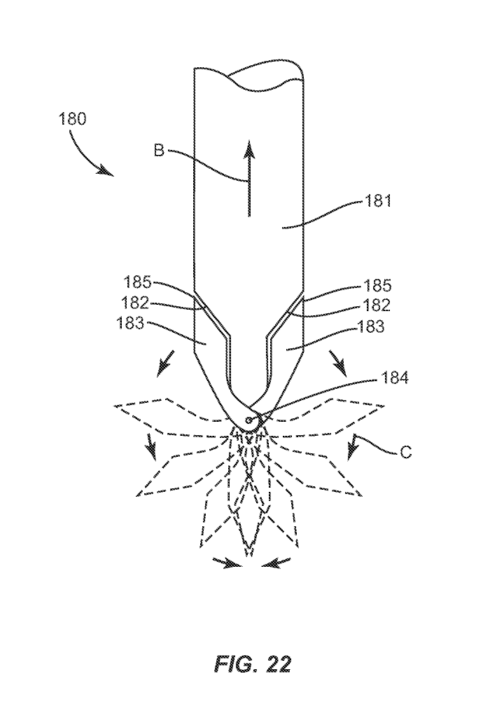

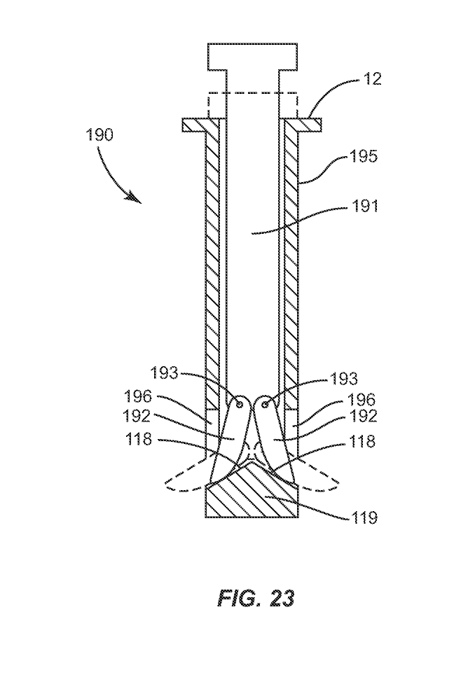

FIG. 22 illustrates another deployable tool 180 that includes a shaft 181 with cut-out sections 182 at the distal end. A pair of wings 183 are connected to the shaft 181 at a hinge 184. Each of the wings 183 includes a sharpened tip 185. The shape of the wings 183 matches the cut-out sections 182 to provide for a reduced width measured perpendicular to the longitudinal axis of the tool 180.

The shaft 181 is inserted into the cannula 10. Movement in this direction causes the wings 183 to remain folded in the retracted orientation. A cylindrical shroud (not illustrated) may also extend around the wings 183 and around the shaft 181 to maintain the wings 183 in the retracted orientation.

Once positioned in the cannula 10 with the tips 185 of the wings 183 aligned at the windows 15, the shroud is moved proximally along the shaft 181 and away from the wings 183. Further, the shaft 181 is moved proximally relative to the cannula 10 causing the tips 185 of the wings 183 to catch on the surrounding material. This causes the wings 183 to pivot at the hinge 184 outward away from the shaft 181. The tips 185 of the wings 183 are positioned to contact against and treat the vertebral members V1, V2. The shaft 181 may be rotated the cannula 10 or moved in a linear motion within the cannula 10 causing the tips 185 to treat the vertebral members V1, V2.

Further proximal movement of the shaft 182 relative to the cannula 10 causes further movement of the wings 183 about the hinge 184 as shown by the dashed lines. The wings 183 move to an overlapping orientation extending outward beyond the shaft 181. This orientation has a reduced width that allows for removal through the cannula 10.

FIG. 23 illustrates another deployable tool 190 with a shaft 191 having a pair of wings 192 connected at hinges 193 at the distal end. An outer housing 195 extends around the shaft 191 and includes windows 196. The outer housing 195 also includes a plug 119 at a distal end. The plug 119 includes angled surfaces 118 that align with the windows 196 in the outer housing 195. The outer housing 195 may also include a tapered tip at the distal end (not illustrated).

In use, the tool 190 is placed in a cannula 10 with the windows 196 on the outer shaft 195 positioned outward beyond the first end 11 of the cannula 10 are at windows 15 in the cannula 10. The shaft 191 is the moved in a distal direction into the outer housing 195. The tips of the wings 192 contact against the angled surfaces 118 causing the wings 192 to pivot outward through the windows 196 in the outer housing 195. This positioning exposes the wings 192 to contact against the vertebral members V1, V2. The tool 190 can then moved as a unit in either or both rotary and linear motions to treat the vertebral members V1, V2.

Once completed, the shaft 191 is moved in a proximal direction relative to the outer housing 195. This movement causes the wings 192 to contact against the edges of the windows 196 and to pivot about the hinges 184 inward into the interior of the outer housing 195. This retracted orientation allows for tool 190 to be removed through the cannula 10.

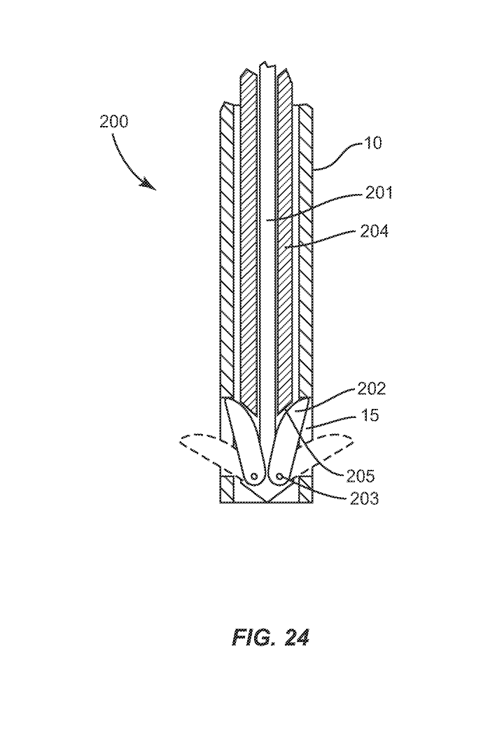

FIG. 24 illustrates a similar tool 200 with wings 202 that are pivotally connected at hinges 203 to a shaft 201. The shaft 201 further extends through an outer housing 204 with a distal end 205 that includes angled surfaces. The wings 202 are positioned outward beyond the distal end 205 of the housing 204.

In use, the shaft 201 with the wings 202 and the housing 204 are inserted in the cannula 10 with the tips of the wings 202 being aligned with the windows 15 in the cannula 10 or outward beyond the first end 11 of the cannula 10. Once positioned, the shaft 201 is moved in a proximal direction relative to the housing 204. This movement causes the tips of the wings 202 to contact against the angled surface on the distal end 205 of the housing 204. Further movement of the shaft 201 in the proximal direction causes the wings 202 to pivot outward from the shaft 201 where they can contact against and treat the vertebral members V1, V2.

Once complete, the shaft 201 is moved in a distal direction relative to the housing 204. The wings 202 contact against edges of the cannula 10 that form the window 15 and pivot to retract inward back towards the shaft 201. In one embodiment, biasing members, such as a coil spring, are attached to the wings 202 to provide a force to return the wings 202 inward back towards the shaft 201.

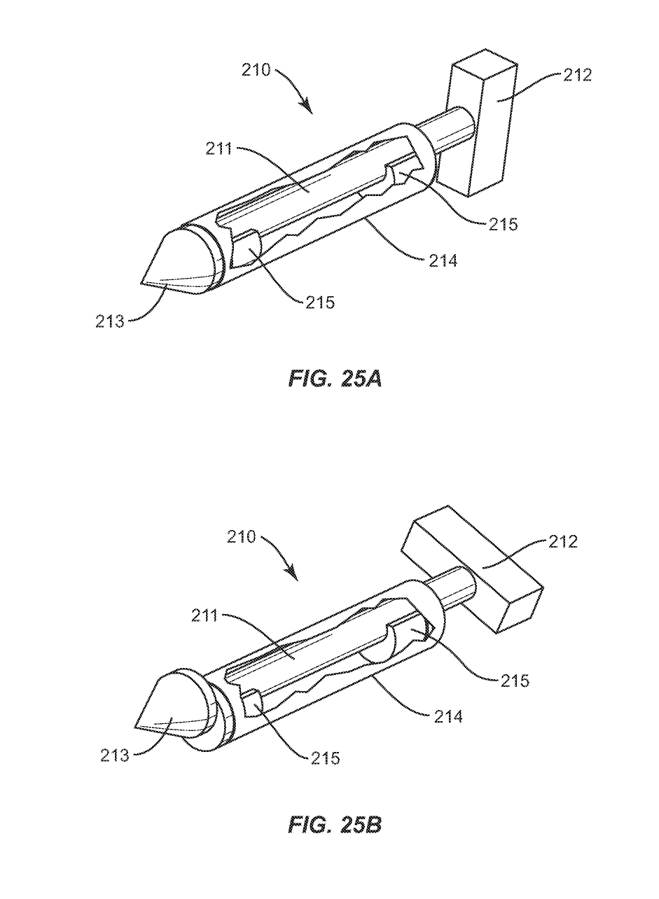

FIGS. 25A and 25B illustrate another deployable tool 210 that includes an elongated shaft 211 with a handle 212 at the proximal end and a tapered tip member 213 at the distal end. A cylindrical housing 214 extends around the shaft with the handle 212 and the tip 213 positioned outward from each end. One or more holders 215 are positioned in the interior of the housing and include receptacles to contact against the shaft 211. The holders 215 radially offset the shaft 211 away from the longitudinal axis of the housing 215. Further, the distal end of the shaft 211 is attached to the tip 213 away from a center of the tip 213. The shaft 211 with the handle 212 and tip 213 are rotatable relative to the housing 215.

FIG. 25A illustrates the tool 210 in a retracted orientation with the tip 213 aligned with the shaft 211. This orientation provides a reduced size for insertion through the cannula 10 and into the facet joint J. Once positioned, rotation of the handle 212 causes the tip 213 to rotate out of alignment with the shaft 211 as illustrated in FIG. 25B. This positioning exposes the tip 213 for contacting against and treating the vertebral members V1, V2. The tip 213 may include one or more cutting edges to facilitate treatment of the vertebral members V1, V2. Once the treatment is complete, the handle 212 is rotated to return the tip 213 into alignment with the housing 214 for removal from the facet joint J.

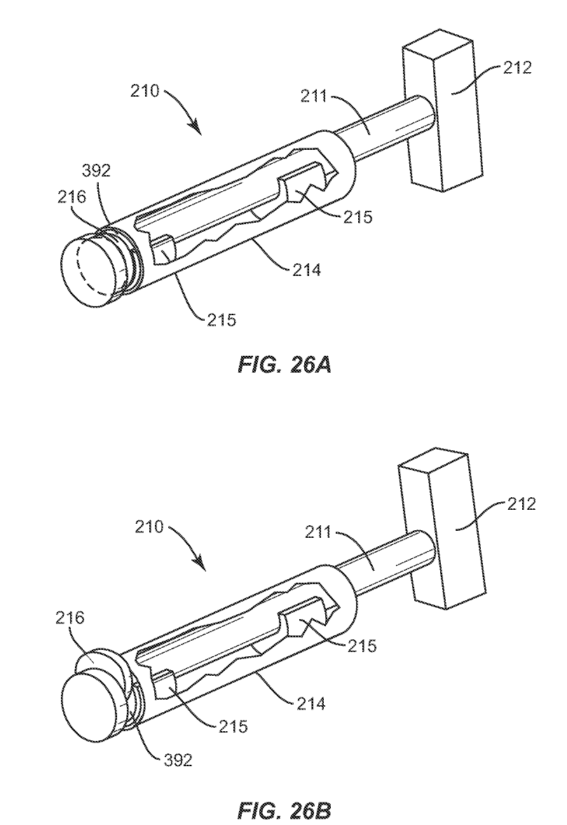

FIGS. 26A and 26B illustrate a similar tool 210 with the housing 214 including a window 392 positioned away from the distal end. A scraper member 216 is attached to the shaft 211 and aligned with the window 392. The scraper 216 includes one or more cutting edges. As with the previous tool 200, the shaft 211 is attached to the scraper member 216 at a point offset from the center.

FIG. 26A illustrates the tool 210 in a first orientation with a reduced size for insertion into the facet joint J. Once positioned, the handle 212 is rotated causing the scraper 216 to rotate out through the window 392. This exposes the cutting edges for contact against and treating of the vertebral members V1, V2. Once the process is completed, the handle 212 is rotated and the scraper 216 is returned to alignment with the housing 214 for removal from the facet joint J.

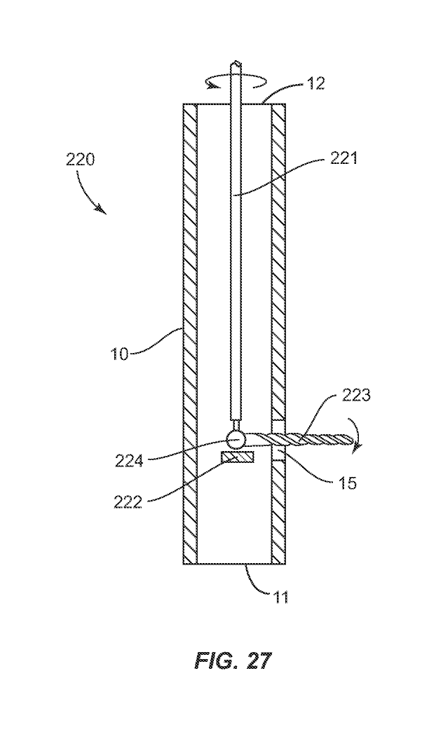

FIG. 27 includes another deployable tool 220 that is used with a cannula 10 having a window 15. The cannula 10 further includes an abutment member 222 positioned in proximity to the window 15. The abutment member 222 may be a floor that extends across the interior of the cannula 10. The tool 220 includes an elongated shaft 221, a drill member 223, and a universal joint 224 that connects the two together. The drill member 223 may include a sharpened tip and helical threads. The universal joint 224 pivotally connects the drill member 223 to the shaft 221, and also transfers rotation from the shaft 221 to the drill member 223.

In use, the shaft 221 and the drill member 223 are substantially aligned in a straight orientation for insertion into the cannula 10. The tool 220 is inserted until the tip of the drill member 223 contacts against the stop 222. Further movement of the shaft 221 causes the drill member 223 to deflect radially outward towards the window 15 due to the universal joint 224. Continued insertion moves the drill member 223 through the window 15 and radially outward beyond the cannula 10. The shaft 221 is rotated with the force being transferred through the universal joint 224 to the drill member 223 to create small holes in the one of the vertebral members V1, V2. Once completed, the cannula 10 is rotated for the drill member 223 to contact against the other vertebral members V1, V2. The process is then repeated. Once the vertebral members V1, V2 have been treated, the shaft 221 is moved proximally upward relative to the cannula 10. This movement causes the drill member 223 to pivot at the universal joint 224 and move back into the interior of the cannula 10 for removal from the facet joint J.

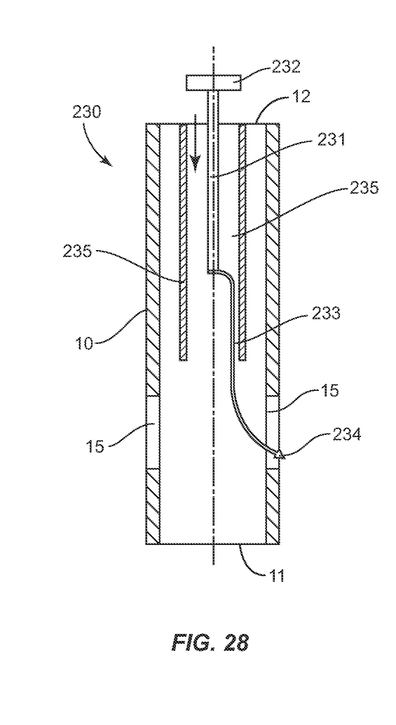

FIG. 28 includes a deployable tool 230 with a short inner sleeve 235 positioned within the interior of the cannula 10. The shorter length positions the distal end of the inner sleeve 235 in proximity to the windows 15. An inner member is positioned within the inner sleeve 235. The inner member includes a shaft 231 with a handle 232, and one or more shape-memory, pre-formed, self deployable arms 233. Each of the arms 233 includes a sharpened tip 234 at the distal end. The arms 233 are initially constrained within the interior of the inner sleeve 235.

In use, the tool 230 is inserted into the facet joint J with the windows 15 facing the two opposing vertebral members V1, V2. The shaft 231 is moved through the inner sleeve 235 with the preformed arms 233 moving beyond the distal end of the inner sleeve 235. The preformed arms 233 extend radially outward away from the longitudinal axis of the cannula 10. Further movement of the shaft 231 moves the arms 233 through the window 15. The arms 233 are constructed such that they will deploy and follow a known trajectory. In embodiments with a single arm 233, the device can be rotated to contact against and treat the opposing vertebral member V1, V2. Once the process is completed, the shaft 231 is moved proximally through the cannula 10. This movement causes the arms 233 to contact against the cannula 10 and inner sleeve 235 and pull radially inward for removal from the facet joint J.

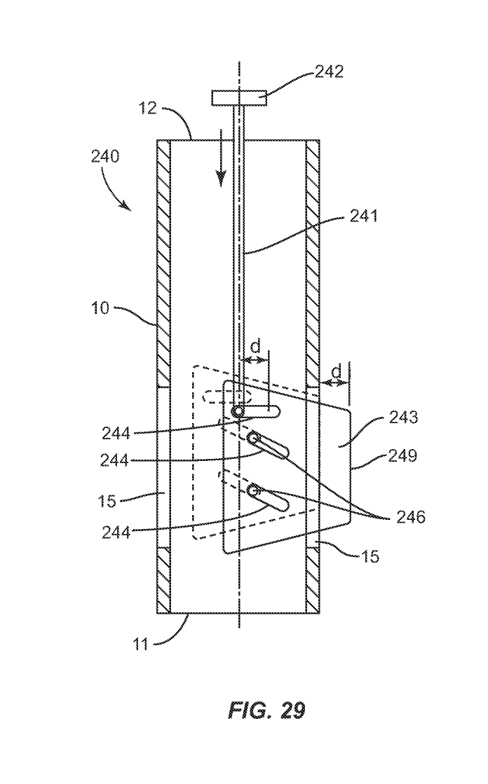

FIG. 29 illustrates another deployable device that includes a member 243 with a cutting edge 249 and one or more slots 244. The slots 244 are positioned at an angle relative to the longitudinal axis of the cannula 10. Pins 246 are positioned in the slots 244 to connect the blade 243 to the cannula 10. An additional slot 244 receives another pin 246 for attaching the blade 243 to a distal end of a shaft 241.

In a first orientation, the blade 243 is positioned within the interior of the cannula 10 (illustrated by dashed lines in FIG. 29). A downward force is applied to the shaft 241. This force causes the blade 243 to move radially with the slots 244 moving around the pins 246. This movement places the cutting edge 249 radially outward beyond the cannula for contact against and treating of one of the vertebral members V1, V2. Once completed, the device can be rotated for treating the other vertebral member V1, V2.

Once the entire process is completed, the shaft 241 is pulled proximally upward through the cannula 10. This movement causes the blade 243 to move over the pins 246 and radially inward into the interior of the cannula 10 for removal from the facet joint J.

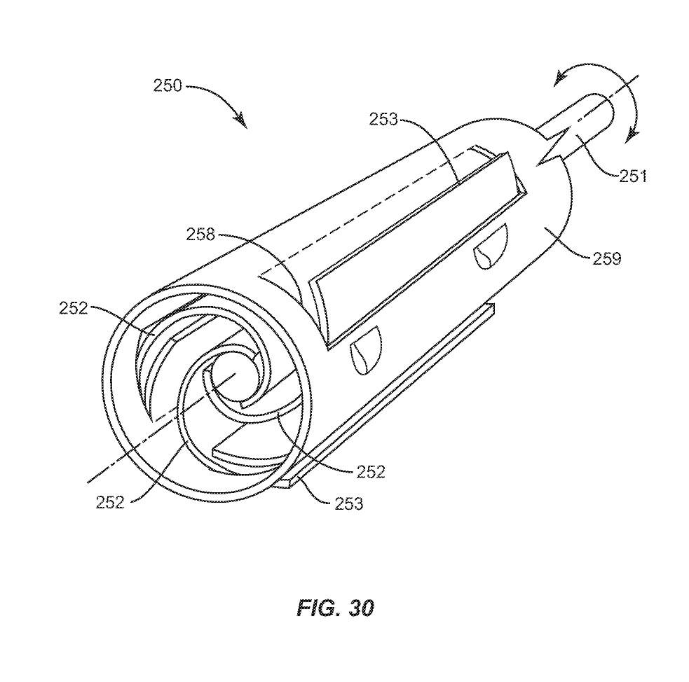

FIG. 30 illustrates another deployable tool 250 that includes a housing 259 with a number of elongated windows 258. A shaft 251 is sized to fit within the interior of the housing 259. A number of scrapers 252 are rolled around the shaft 251 with a first side attached to the shaft 251 and an opposing second side including a cutting edge. Each of the scrapers 252 may be formed from a sheet of rolled spring steel.

In use, the tool 250 is in the first orientation and is inserted through the cannula 10 and outward beyond the first end 11. In the first orientation, the scrapers 252 are rolled around the shaft 251 and positioned within the interior of the outer housing 259. The shaft 251 is then rotated in a first direction that causes the exposed second sides of the scrapers 252 that are aligned with the windows 258 to extend outward through the windows 258 and contact against the vertebral members V1, V2. The amount that the scrapers 252 extend outward through the windows 258 depends upon the amount of rotation of the shaft 252.

Once the treatment is complete, the shaft 251 is rotated in a second direction. This causes the scrapers 252 to move inward through the windows 258 and into the interior of the outer housing 259 for removal through the cannula 10. The outer housing 259 may include embossed features that mate with similar features on the scrapers 252 to prevent the scrapers 252 from being retracted too far inward into the housing 259.

The various tools 20 form a space in the facet joint J to receive osteogenic material. The process generally includes a minimally invasive approach to the facet joint J. Initially, an incision is made in the patient and a series of tissue dilators of increasing size are inserted to create a larger opening for the cannula 10. The cannula 10 is then inserted through the soft patient tissue with the first end 11 positioned at the facet joint J and the second end 12 remaining outward beyond the patient.

After the cannula 10 is positioned at the facet joint J, one of the tools 20 is inserted through the cannula 10. The tool 20 may include a length with the treating section 24 of the tool extending outward beyond the first end 11 of the cannula 10 or through a window in the cannula 10. The treating section 24 is moved through the cannula 10 and against one or both vertebral members V1, V2. Once the treatment is complete, the tool 20 is moved in the opposing direction upward through the cannula 10. The advancement of the tool 20 may occur as a continuous motion in a single axial direction into the facet joint J. Alternatively, the advancement may occur in short oscillating steps with forward and backward motion of the tool 20 against the vertebral members V1, V2.

With a rotary tool 20, the tool 20 may be rotated in just one direction during movement through the cannula 10 and against the vertebral members V1, V2. The tool 20 may also be configured for rotation in a second direction with a first amount of axial movement occurring during rotation in a first direction, and a second amount occurring during rotation in a second direction. In one embodiment, the tool 20 is rotated in a first direction while being inserted into the facet joint J, and rotated in a second direction when being removed from the facet joint J.

In the various embodiments, multiple tools 20 may be used for forming the space in the facet joint J. A first tool 20 may be initially inserted into the facet joint J to create a space having a first size and/or shape. Subsequently, a second tool 20 is inserted to enlarge or further shape of size and/or shape of the space. Further tools 20 may also be inserted through the cannula 10 and into the space as necessary.

In some embodiments, the tools 20 contact against and treat both vertebral members V1, V2 simultaneously to create the graft space. In other embodiments, the tools 20 may contact and treat a single vertebral member V1, V2 at a time. A first tool 20 is inserted to treat the first vertebral member V1. This tool 20 is then either rotated or otherwise adjusted to treat and contact the second vertebral member V2, or one or more additional tools 20 are used for treating the second vertebral member V2. In some embodiments, the graft space is formed by treating just one of the vertebral members V1, V2.

In some embodiments, a single cannula 10 is used during the entire process of treating the facet joint J. Other embodiments include the use of two or more different cannulas 10. A first cannula 10 is inserted into the facet joint J for one or more of the process steps. The first cannula 10 is removed, and a second cannula 10 is inserted for one or more subsequent process steps. The second cannula 10 may include a different physical property that is necessary for one or more of the steps. This may include a specific cross-sectional shape or size necessary to guide a tool 20 into the facet joint J. Additional cannulas 10 may be inserted as necessary to guide the various tools 20 and create the required graft space.

The various tools 20 are each configured to treat one or both of the vertebral members V1, V2 during use. The treatment may include removal of various amounts of material from one or both of the vertebral members. The treatment may decorticate the inner surfaces of the facet joint J to maximize the bony contact with the osteogenic material.

After the graft space has been formed in the facet joint J, the process further includes implanting osteogenic material into the space. In one embodiment, the osteogenic material fills the entire facet joint J to increase the chances for fusion. This may include delivery through a rectangular or elliptical shaped cannula 10 as illustrated in FIG. 31. In one specific embodiment for fusion of the lumbar spine which includes the facet joins being approximately 10-15 mm tall, the use of an elongated osteogenic material is beneficial.

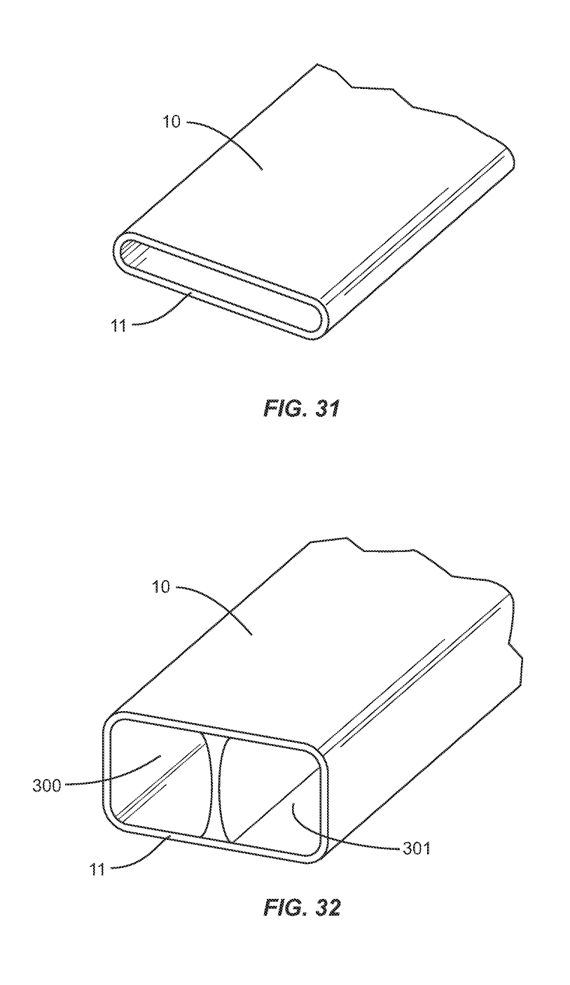

In some embodiments, a single piece of osteogenic material is placed in the facet joint J. This may be implanted using a cannula 10 with a single interior lumen. Other embodiments may include multiple separate pieces of osteogenic material being positioned within the facet joint J. These embodiments may include a cannula 10 with multiple lumens. FIG. 32 includes a cannula 10 with first and second separate lumens 300, 301. A first osteogenic material is inserted through the first lumen 300, and a second osteogenic material is inserted through the second lumen 301. Other embodiments may include additional lumens as necessary for placement of different osteogenic materials. Further, the cross-sectional shapes and sizes of the different lumens within the cannula may be the same or different depending upon the application.

In some embodiments, the osteogenic material may be inserted into the facet joint over a guide wire. These embodiments include the osteogenic material including an opening to extend over the guide wire.

A cage may be positioned in the facet joint J to receive the osteogenic material. The cage includes an interior space sized to receive the osteogenic material. In one embodiment, the cage is introduced after treatment of the facet joint J. In another embodiment, the cage is used also as the treating process for preparing the facet joint J. The movement of the cage across the vertebral members V1, V2 provides treatment for receiving the osteogenic material.

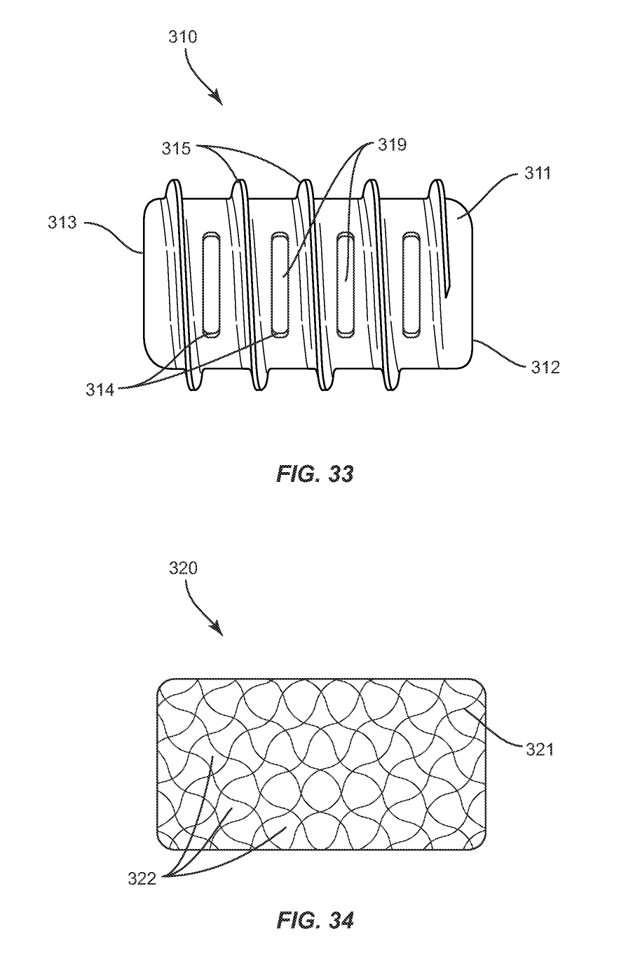

FIG. 33 includes one embodiment of a cage 310 that includes threads 315 for entry into the facet joint J. The threads 315 secure the cage 310 in the facet joint J and may also further treat the vertebral members V1, V2 for preparation of the osteogenic material. The first end 312 may be tapered to facilitate insertion into the facet joint J. The cage 310 includes an open interior 319 for receiving the osteogenic material. One or more openings 314 are positioned about the cage 310 that extend into the interior 319. The exterior of the cage 310 may include an abrasive finish such as a sputter coating to further treat the vertebral members V1, V2 to encourage bony ingrowth. The cage 310 may be constructed from a variety of materials, including but not limited to titanium, plastic, and cortical bone from processed bone allograft.

The osteogenic material may be placed into the cage 310 prior to insertion into the facet joint J. Alternatively, the cage 310 may be positioned into the facet joint J prior to insertion of the osteogenic material.

FIG. 34 includes a mesh casing 320 that is introduced into the facet joint J to contain the osteogenic material. The mesh comprises a plurality of fibers 321 that are woven together having intermediate openings 322. The fibers 321 may include a variety of materials, including but not limited to titanium, steel, or other metal wire, PET fabric, polypropylene fabric, polyethylene fabric, and/or steel, titanium or other metal wire. The mesh casing 320 may be inserted into the facet joint J after treatment of the vertebral members V1, V2, or as a way to treat the vertebral members. Further, the osteogenic material may be placed in the interior of the casing 320 prior to or after insertion into the facet joint J.

Osteogenic materials include, without limitation, autograft, allograft, xenograft, demineralized bone, cancellous bone graft, cortical bone graft, synthetic and natural bone graft substitutes, such as bioceramics and polymers, and osteoinductive factors. The osteogenic compositions may include an effective amount of a bone morphogenetic protein (BMP), demineralized bone matrix (DBM), transforming growth factor f31, insulin-like growth factor, platelet-derived growth factor, fibroblast growth factor, LIM mineralization protein (LMP), and combinations thereof or other therapeutic or infection resistant agents, separately or held within a suitable carrier material.

It is envisioned that system 10 may be used in any existing surgical method or technique including open surgery, mini-open surgery, minimally invasive surgery and percutaneous surgical implantation, whereby the facet joint is accessed through a micro-incision, or sleeve that provides a protected passageway to the area. Once access to the surgical site is obtained, the particular surgical procedure employing system 10 is performed for treating the spinal disorder. The system 10 may also be used for intervertebral disc applications. In these applications, the sizes of the various cannulas 10, tools 20, and handles 30 may be enlarged to accommodate the relatively larger anatomy.

The various methods described above generally include a tool 20 used with a cannula 10. In addition, the tools 20 may be used without a cannula 10. By way of example, the deployable tool 250 may be used independently of a cannula 10.

The various tools 20 include elongated shapes. The shapes may be substantially straight, or may be curved or bent at a point between the opposing ends.

The instrument 10 may be used during surgical procedures on living patients. The instrument 10 may also be used in a non-living situation, such as within a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A method of fusing a facet joint formed by first and second vertebral members comprising:
percutaneously inserting a cannula into a patient with a first end of the cannula at the facet joint and a second end of the cannula positioned outward from the patient;
inserting a first tool through the cannula and moving a treating section of the tool outward beyond the first end of the cannula;
moving the treating section of the first tool and contacting against at least one of the vertebral members and enlarging the facet joint;
removing the first tool through the second end of the cannula;
inserting a second tool into the second end of the cannula and moving a treating section of the second tool outward beyond the first end of the cannula;
moving the treating section of the second tool and enlarging the facet joint;
removing the second tool through the second end of the cannula; and
inserting an osteogenic material into the enlarged facet joint.

2. The method of claim 1, further comprising contacting a cutting edge positioned on the cannula against the first vertebral member and removing portions of the first vertebral member and enlarging the facet joint.

3. The method of claim 2, further comprising rotating the cannula and moving the cutting edge against the second vertebral member and removing portions of the second vertebral member.

4. The method of claim 2, further comprising moving the cannula in an oscillating linear manner and contacting the cutting edge against the first vertebral member and removing the portions of the first vertebral member.

5. The method of claim 1, further comprising simultaneously contacting the working section of the second tool against both of the first and second vertebral members and removing sections of both of the first and second vertebral members.

6. The method of claim 1, further comprising contacting just the first vertebral member with the working section of the first tool and removing sections of just the first vertebral member.

7. The method of claim 6, further comprising rotating the first tool about 180° within the cannula and contacting just the second vertebral member with the working section of the first tool and removing sections of just the second vertebral member.

8. The method of claim 1, wherein inserting the osteogenic material into the enlarged facet joint includes inserting a second cannula into the patient that has a non-circular cross-sectional shape and moving the osteogenic material through the second cannula and into the facet joint.

9. The method of claim 1, wherein inserting the osteogenic material into the enlarged facet joint includes inserting a second cannula into the patient that has first and second separate lumens and moving a first piece of the osteogenic material through the first lumen and into the enlarged facet joint and moving a separate second piece of the osteogenic material through the second lumen and into the enlarged facet joint.

10. A method of fusing a facet joint formed by first and second vertebral members comprising:
inserting a cannula into a patient with a first end of the cannula at the facet joint and a second end of the cannula spaced away from the facet joint, the first end including a cutting edge;
moving the cannula and contacting the cutting edge against the first and second vertebral members and removing first portions of the first and second vertebral members with the cutting edge;
inserting a tool through the cannula and moving a treating section of the tool outward beyond the first end of the cannula;
moving the treating section of the tool against the first and second vertebral members and removing second sections of the first and second vertebral members;
removing the tool through the second end of the cannula; and
inserting an osteogenic material into the facet joint and against the first and second vertebral members.

11. The method of claim 10, wherein moving the treating section of the tool against the first and second vertebral members includes rotating the treating section and removing the second portions of the first and second vertebral members during the rotation of the treating section against the first and second vertebral members.

12. The method of claim 10, wherein moving the treating section of the tool against the first and second vertebral members includes linearly oscillating the treating section and removing the second portions of the first and second vertebral members during the oscillating linear movement of the treating section against the first and second vertebral members.

13. The method of claim 10, wherein moving the cannula and contacting the cutting edge against the first and second vertebral members includes rotating the cannula and removing the first portions of the first and second vertebral members during rotation of the cutting edge against the first and second vertebral members.

14. The method of claim 10, further comprising simultaneously moving the cutting edge of the cannula against the first and second vertebral members and removing the first portions of the first and second vertebral members and moving the treating section of the tool against the first and second vertebral members and removing the second sections of the first and second vertebral members.

15. The method of claim 10, further comprising moving a cutting edge formed along a window in the cannula against one of the first and second vertebral members, the window being spaced away from the first end of the cannula.

16. The method of claim 10, wherein inserting the osteogenic material into the facet joint and against the first and second vertebral members includes inserting the osteogenic material into a holding member positioned in the facet joint.

17. A method of fusing a facet joint formed by first and second vertebral members comprising:
percutaneously inserting a cannula into a patient with a first end of the cannula at the facet joint and a second end of the cannula positioned outward from the patient;
inserting a tool into the second end of the cannula with a treating section of the tool in a retracted orientation;
moving the treating section along the cannula and away from the second end;
deploying the tool and moving the treating section of the tool to a second extended orientation;
contacting the treating section of the tool in the extended orientation against at least one of the first and second vertebral members and removing sections of at least one of the first and second vertebral members;
returning the tool to the retracted orientation; and
removing the tool through the second end of the cannula while the tool is in the retracted orientation.

18. The method of claim 17, further comprising moving the treating section outward beyond the first end of the cannula prior to deploying the tool to the second extended orientation.

19. The method of claim 17, further comprising aligning the treating section of the tool with a window in the cannula and moving the treating section through the window when deploying the tool.

20. The method of claim 17, further comprising inserting a holding member into the facet joint with the holding member including a hollow interior to hold osteogenic material.
